(19) United States
(12) Patent Application Publication
(10) Pub. No.: US 2016/0143946 A1
(43) Pub. Date: May 26, 2016

(54) COMBINATION THERAPIES FOR TREATING CANCER

(71) Applicant: Arnold I. Chin, Los Angeles, CA (US)

(72) Inventor: Arnold I. Chin, Los Angeles, CA (US)

(21) Appl. No.: 14/950,285

(22) Filed: Nov. 24, 2015

Related U.S. Application Data
(60) Provisional application No. 62/083,576, filed on Nov. 24, 2014.

(51) Int. Cl. A61K 33/24; A61K 45/06; A61K 31/16

(52) U.S. Cl. CPC A61K 33/24; A61K 31/16; A61K 45/06

(57) ABSTRACT

Aspects described herein provide methods of treating cancer by administering a histone-deacetylase inhibitor with a chemotherapeutic. In some embodiments, the histone-deacetylase inhibitor is AR-42. In some embodiments, the chemotherapeutic is cisplatin.

### CROSS-REFERENCE TO RELATED APPLICATIONS

This application claims the benefit of U.S. Provisional Application No. 62/083,576, filed on Nov. 24, 2014, the entire contents of which are incorporated by reference herein in their entirety.

### BACKGROUND

Cancer includes a number of diseases characterized by the uncontrolled division of abnormal cells. Cancer may initially arise in virtually any tissue or organ in the body and forms as a result of a complex interaction of both innate genetic factors and environmental factors, such as one's diet or exposure to radiation, toxins, and the like. Despite advances in medicine and the understanding of the molecular basis of cancer, the exact causes of any given type of cancer are largely unknown, especially in a particular individual. Given this lack of knowledge, it is not surprising that it remains highly difficult to find effective cancer treatments.

Finding effective treatments is also challenging because cancer often develops resistance to various therapeutic strategies. In addition, effective means for treating cancer become an even greater challenge in view of the capacity for certain types of cancers to to spread from their primary source. This process, called metastasis, enables cancer cells to spread to other vital parts of the body through the blood and lymph systems. Some experts estimate that only a single cell in a million can survive long enough to help form a metastatic tumor. These odds are thought to be attributable to the challenges metastasized cells face in the destination tissue, including lodging in the destination tissue, overcoming local immune defenses, and acquiring their own blood supply and nutrients through the process of angiogenesis. Nevertheless, metastasis remains a key reason why effective cancer treatments are difficult to develop.

Existing cancer therapies today include multiple different ablation techniques such as surgical procedures; cryogenic or heat methods on the tissue, ultrasound, radiofrequency, and radiation; chemical methods such as pharmaceuticals, cytotoxic agents, monoclonal antibodies; or transarterial chemo immobilization (TACE), and combinations thereof pursuant to specific regimens based on the specific type and stage of cancer under treatment. However, these therapies often incur substantially high costs. In addition, current treatment options are often highly invasive, are associated with significant toxicities, and result in an overall poor quality of life for patients.

Standard of care cancer therapies typically couple surgical removal of the affected tissue with chemotherapy or radiation treatments. Many standard chemotherapeutics are delivered through the blood, e.g., systemically, which can be achieved by various routes such as oral, intravenous and/or gastrointestinal delivery. However, toxicity is a major drawback associated with systemically delivered chemotherapeutic drugs. Standard of care surgical treatments often have a tendency to introduce problems, including dislodgement of cancer cells into the blood and/or lymph systems, which creates an opportunity for cancer cells to metastasize to other sites in the body and form additional tumors. When surgery is not possible, the accepted treatment for cancer is to use radiation or chemotherapy. But survival rates for inoperable cancer are low when compared to the survival rate for cancers that are surgically removed prior to chemotherapy or radiation. Improved methods of treating cancer are needed to improve survival rates and reduce side effects (e.g., the toxicity of a chemotherapy).

### SUMMARY OF THE DISCLOSURE

The present invention provides a method of treating cancer in a subject in need thereof by administering to the subject (e.g., orally or parenterally, e.g., intravenously) a histone-deacetylase inhibitor and a chemotherapeutic, e.g., in effective amounts. In certain preferred embodiments, the histone-deacetylase inhibitor is AR-42 (Compound I).

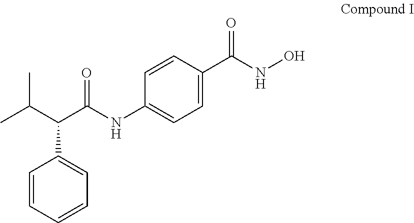 [SMILES: CC(C)[C@H](C(=O)Nc1ccc(C(=O)NO)cc1)c1ccccc1]

In some embodiments, administering the chemotherapeutic comprises administering aminoglutethimide, amsacrine, anastrozole, asparaginase, bcg, bicalutamide, bleomycin, bortezomib, buserelin, busulfan, campothecin, capecitabine, carboplatin, carfilzomib, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pemetrexed, pentostatin, perifosine, plicamycin, pomalidomide, porfimer, procarbazine, raltitrexed, rituximab, sorafenib, streptozocin, sunitinib, suramin, tamoxifen, temozolomide, temsirolimus, teniposide, testosterone, thalidomide, thioguanine, thiotepa, titanocene dichloride, topotecan, trastuzumab, tretinoin, vinblastine, vincristine, vindesine, or vinorelbine. Preferably, administrating the chemotherapeutic may comprise administering pemetrexed or, more preferably, cisplatin. HDAC inhibitors include compounds disclosed in U.S. Pat. No. 8,318,808, incorporated by reference herein in its entirety. In some embodiments, the cancer is ovarian cancer, cervical cancer, brain cancer, lung cancer, skin cancer, colorectal cancer, esophageal cancer, breast cancer, prostate cancer, leukemia, lymphoma, multiple myeloma, bone cancer, pancreatic cancer, bladder cancer, endometrial cancer, kidney cancer, liver cancer, eye cancer, pituitary cancer, testicular cancer, and stomach cancer. Preferably, the cancer is bladder cancer, lung cancer, breast cancer, prostate cancer, lymphoma, or leukemia. More preferably, the cancer is bladder cancer. In some embodiments, administering a chemotherapeutic comprises administering a low-dose chemotherapy. In some embodiments, the chemotherapeutic is administered at an amount that is less than the standard amount. The chemotherapeutic may be administered at an amount that reduces the risk or severity of side effects associated with the chemotherapeutic relative to the standard amount. In some embodiments, the patient has a disease or condition that is a contraindication for standard-dose chemotherapy. In some embodiments, the patient has reduced creatinine clearance or neutropenia. In some embodiments, the combination of administering the histone-deacetylase inhibitor and the chemotherapeutic is more efficacious than administering either treatment alone. In some embodiments, the histone-deacetylase inhibitor increases the apoptosis or autophagy of cancer cells. The subject may be a mammal (e.g., a human).

In another aspect, the invention provides a pharmaceutical composition comprising a histone-deacetylase inhibitor and a chemotherapeutic. The pharmaceutical composition may be formulated for any route of administration, e.g., oral or parenteral administration. In some embodiments, the pharmaceutical composition is self-administered. The histone-deacetylase inhibitor may be AR-42. The chemotherapeutic may comprise aminoglutethimide, amsacrine, anastrozole, asparaginase, bcg, bicalutamide, bleomycin, bortezomib, buserelin, busulfan, campothecin, capecitabine, carboplatin, carfilzomib, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pemetrexed, pentostatin, perifosine, plicamycin, pomalidomide, porfimer, procarbazine, raltitrexed, rituximab, sorafenib, streptozocin, sunitinib, suramin, tamoxifen, temozolomide, temsirolimus, teniposide, testosterone, thalidomide, thioguanine, thiotepa, titanocene dichloride, topotecan, trastuzumab, tretinoin, vinblastine, vincristine, vindesine, or vinorelbine. Preferably, the chemotherapeutic comprises cisplatin or pemetrexed, most preferably cisplatin.

### DESCRIPTION OF THE FIGURES

FIG. 1 shows an exemplary characterization of a bladder cancer cell line in response to cisplatin and HDAC inhibitors. SW780 and HT1376 bladder cancer cell lines stained with anti-CD44 and anti-CD49f were examined by flow cytometry. Cells were treated with cisplatin, AR-42, sodium butyrate, valproic acid, and trichostatin A at concentrations indicated for 24 hours and cell viability was determined by MTT incorporation. The table denotes IC50s for SW780 and HT1376 cells to listed agents.

FIG. 2 shows the percent survival of SW780 and HT1376 cells treated, in an exemplary experiment, with cisplatin, AR-42, a combination cisplatin and AR-42 at a 5:1 molar ratio, a combination cisplatin and AR-42 in a 20:1 ratio, as assessed by MTT incorporation. Isobolograms were used to calculate combination indices to indicate synergy at IC50 and IC90, whereby CI<1 denotes synergy.

FIG. 3 shows that the combination of AR-42 and cisplatin is a synergistic treatment for CD44⁺CD49f⁺ bladder cancer cells and depicts an exemplary % survival of SW780 bladder cancer cells sorted into CD44⁻CD49f⁻, CD44⁻CD49f⁻, CD44⁻CD49f⁻, and CD44⁺CD49f⁺ populations by flow cytometry and treated with cisplatin, AR-42, or a combination cisplatin and AR-42. The percent survival was assessed by 3-(4,5-dimethylthiazol-2-yl)-2,5-diphenyltetrazolium bromide (MTT) incorporation.

FIG. 4 shows the % survival of SW780 cells that were treated simultaneously, in an exemplary experiment, with cisplatin and AR-42 for 24 hours, sequentially with cisplatin for 24 hours followed by AR-42 for 24 hours, and sequentially with AR-42 for 24 hours followed by cisplatin for 24 hours. Cells were assessed by flow cytometry for apoptosis, by Annexin V staining, and autophagy, by LC3 staining.

FIG. 5 shows that the in vivo administration of combined cisplatin and AR-42 decreases tumor size relative to cisplatin or AR-42 alone in an exemplary experiment. SW780 (1×10⁵) cells were combined with human fetal bladder mesenchymal cells (1×10⁶) in Matrigel, subcutaneously implanted in Nod scid IL-2 receptor gamma knockout animals, and assessed for tumor size. Experimental results were histologically assessed. Tumor volume was calculated by (length×width×height)×0.4. Box plots denote the mean tumor volume calculated from 6 tumors in each group.

### DETAILED DESCRIPTION OF THE DISCLOSURE

Aspects provided herein relate to synergy between HDAC inhibitors and other chemotherapeutic agents (e.g., platinum-based therapeutic agents) when administered in combination to cancer cells. This combination treatment reduces the survival of cancer cells in cancer cell lines and reduces tumor volumes in animal models of cancers. Accordingly, certain aspects relate to the administration of a HDAC inhibitor and a chemotherapeutic, such as a platinum-based chemotherapeutic to treat a cancer cell and/or to treat a tumor.

Histone acetylation and deacetylation play a critical role in gene regulation modified by the enzymes histone acetyltransferase and HDAC. Histone deacetylation inhibits transcription by removing acetyl groups from histones and increasing histone-DNA affinity. Thus, HDAC inhibitors, which induce epigenetic changes through modification of both histone and non-histone proteins, have emerged as a novel class of cancer therapeutics.

Some aspects provide methods for inducing apoptosis, causing cell death, and/or inducing autophagy of cells. In one aspect, the method comprises administering an effective amount of a HDAC inhibitor and a chemotherapeutic to a cell or subject in need thereof. An effective amount of HDAC inhibitor and a chemotherapeutic is referred to as a combination treatment herein, and the HDAC inhibitor and chemotherapeutic of the combination treatment may be administered simultaneously or sequentially. The method of the disclosure is particularly useful in inducing apoptosis or autophagy of abnormal cells but not normal cells. Abnormal cells include any type of cell that is causative of or involved in a disease or condition and wherein it is desirable to modulate or inhibit the proliferation of the abnormal cell to treat the disease or condition. Examples of abnormal cells include malignant or cancerous cells, such as tumor cells. Abnormal cells may be the cells of a cell line, for example, abnormal cells may be the cells of a cancer cell line. In some embodiments, the abnormal cells are in a subject. For example, the cells may be the tumor cells of an animal model of cancer, preferably a human subject.

In some embodiments, the combination treatment of the disclosure is effective at killing cancer cells while at the same time they do not kill normal cells. In some embodiments, the combination treatment is more effective at killing cancer cells while at the same time they are less effective at killing normal cells, i.e., the combination treatment selectively kills cancer cells. These properties make the combination treatment of the disclosure useful as an anti-cancer treatment. Accordingly, in some embodiments, the present invention provides a method of causing apoptosis or autophagy of a cancer cell, comprising administering a combination treatment of the disclosure to a cell or subject in need thereof.

Cancer cells that can be treated with a combination treatment of the disclosure may be any type of cancer including, but not limited to, hematopoietic malignancies, including leukemias, lymphomas, and myelomas as well as other types of cancer including sarcomas, carcinomas, melanomas, adenomas, nervous system cancers, and genitourinary cancers. Examples of leukemias include acute lymphoblastic leukemia (ALL), acute myelocytic leukemia (AML), acute myelomonocytic leukemia (AMML), chronic myeloid leukemia (CML), chronic lymphocytic leukemia (CLL) and juvenile myelo-monocytic leukemia (JMML). In some embodiments, the combination treatment of this disclosure are useful in treating a cancer selected from, but not limited to, ovarian cancer, cervical cancer, brain cancer, lung cancer, skin cancer, colorectal cancer, esophageal cancer, breast cancer, prostate cancer, leukemia, multiple myeloma, bone cancer, pancreatic cancer, bladder cancer, endometrial cancer, kidney cancer, liver cancer, eye cancer, pituitary cancer, testicular cancer, and stomach cancer. In some embodiments, the cancer is the cancer is bladder cancer, lung cancer, breast cancer, prostate cancer, lymphoma, or leukemia. In some embodiments, the cancer is bladder cancer.

In addition to the above-mentioned therapeutic uses, the combination treatment of the disclosure is also useful in diagnostic assays, screening assays, and as research tools.

### Compositions and Modes of Administration

In some embodiments (such as the uses described above), the compounds of the disclosure are formulated into pharmaceutical compositions for administration to subjects (such as human subjects) in a biologically compatible form suitable for administration in vivo. Accordingly, in another aspect, aspects disclosed herein provide a pharmaceutical composition comprising a compound of the disclosure in admixture with a suitable diluent or carrier. Such a composition is useful for treating the conditions described herein.

The compositions containing the compounds of the disclosure can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington: The Science and Practice of Pharmacy, Pharmaceutical Press, Gurnee, Ill., USA, 22^{nd} ed., 2012). On this basis, the compositions include, albeit not exclusively, solutions of the substances in association with one or more pharmaceutically acceptable vehicles or diluents, and contained in buffered solutions with a suitable pH and iso-osmotic with the physiological fluids.

The compounds disclosed herein may be used in treating the conditions described herein, in the form of the free base, salts (preferably pharmaceutically acceptable salts), solvates, hydrates, prodrugs, isomers, or mixtures thereof. All forms are within the scope of the disclosure. Acid addition salts may be formed and provide a more convenient form for use; in practice, use of the salt form inherently amounts to use of the base form. The acids which can be used to prepare the acid addition salts include preferably those which produce, when combined with the free base, pharmaceutically acceptable salts, that is, salts whose anions are non-toxic to the subject organism in pharmaceutical doses of the salts, so that the beneficial properties inherent in the free base are not vitiated by side effects ascribable to the anions.

Pharmaceutically acceptable salts within the scope of the disclosure include those derived from the following acids; mineral acids such as hydrochloric acid, sulfuric acid, phosphoric acid and sulfamic acid; and organic acids such as acetic acid, citric acid, lactic acid, tartaric acid, malonic acid, methanesulfonic acid, ethanesulfonic acid, benzenesulfonic acid, p-toluenesulfonic acid, cyclohexylsulfamic acid, quinic acid, and the like.

Pharmaceutically acceptable carriers that may be used in compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose based substances, polyethylene glycol, sodium carboxymethylcellulose, polyacrylates, waxes, polyethylene polyoxypropylene block polymers, polyethylene glycol, and wool fat.

In accordance with the methods of the disclosure, the described compounds may be administered to a patient in a variety of forms depending on the selected route of administration, as will be understood by those skilled in the art. The compositions of the disclosure may be administered orally or parenterally.

Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

In certain embodiments, pharmaceutical compositions suitable for parenteral administration may comprise the compound of the present disclosure in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

A composition comprising a compound of the present disclosure may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In certain embodiments of the disclosure, compositions comprising a compound of the present disclosure can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and the like, each containing a predetermined amount of the compound of the present disclosure as an active ingredient.

In solid dosage forms for oral administration (capsules, tablets, pills, dragees, powders, granules, and the like), one or more compositions comprising the compound of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the compound of the present disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, salts and/or prodrugs thereof, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

A person skilled in the art would know how to prepare suitable formulations. Conventional procedures and ingredients for the selection and preparation of suitable formulations are described, for example, in Remington: The Science and Practice of Pharmacy (22^{nd} ed.) and in The United States Pharmacopeia: The National Formulary (USP 38 NF 33) published in 2015.

The pharmaceutical forms suitable for injectable use include sterile aqueous solutions or dispersion and sterile powders for the extemporaneous preparation of sterile injectable solutions or dispersions.

The compounds of the disclosure may be administered to a subject in need thereof alone or in combination with pharmaceutically acceptable carriers, as noted above, the proportion of which is determined by the solubility and chemical nature of the compound, chosen route of administration and standard pharmaceutical practice.

The dosage of the compounds and/or compositions of the disclosure can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. The compounds of the disclosure may be administered initially in a suitable dosage that may be adjusted as required, depending on the clinical response. To calculate the human equivalent dose (HED) from a dosage used in the treatment of age-dependent cognitive impairment in rats, the formula HED (mg/kg)=rat dose (mg/kg)×0.16 may be employed (see Estimating the Safe Starting Dose in Clinical Trials for Therapeutics in Adult Healthy Volunteers, December 2002, Center for Biologics Evaluation and Research). For example, using that formula, a dosage of 10 mg/kg in rats is equivalent to 1.6 mg/kg in humans. This conversion is based on a more general formula HED=animal dose in mg/kg×(animal weight in kg/human weight in kg)^{0.33}. Similarly, to calculate the HED from a dosage used in the treatment in mouse, the formula HED (mg/kg)=mouse dose (mg/kg)×0.08 may be employed (see Estimating the Safe Starting Dose in Clinical Trials for Therapeutics in Adult Healthy Volunteers, December 2002, Center for Biologics Evaluation and Research).

The HDAC inhibitor and the other chemotherapeutic may be administered as a single formulation or in separate formulations, either simultaneously or sequentially, according to the methods of the disclosure.

The HDAC inhibitor may be abexinostat (PCI-24781), belinostat (PXD101), chidamide (HBI-8000), givinostat, entinostat (MS-275), mocetinostat (MGCD0103), panobinostat (LBH589), phenylbutyrate, pyroxamide, quisinostat (JNJ-26481585), resminostat (4SC-201), romidepsin (Istodax, FR901228), sodium butyrate, trichostatin A, valproic acid, vorinostat (SAHA), 4SC-202, ACY-1215, AR-42, ATRA, CUDC-101, DAC60, BML-210, M344, ME-344, NVP-LAQ-824, CG200745, CHR-3996, CHR-2845, SB939, ITF2357, R306465 (JNJ16241 199), or a pharmaceutically acceptable salt, hydrate, solvate or stereoisomer thereof. In some embodiments, the HDAC inhibitor is AR-42. In some embodiments, the HDAC inhibitor is a Class I, Class II, Class III, or Class IV inhibitor.

The chemotherapeutic may comprise aminoglutethimide, amsacrine, anastrozole, asparaginase, bcg, bicalutamide, bleomycin, bortezomib, buserelin, busulfan, campothecin, capecitabine, carboplatin, carfilzomib, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pemetrexed, pentostatin, perifosine, plicamycin, pomalidomide, porfimer, procarbazine, raltitrexed, rituximab, sorafenib, streptozocin, sunitinib, suramin, tamoxifen, temozolomide, temsirolimus, teniposide, testosterone, thalidomide, thioguanine, thiotepa, titanocene dichloride, topotecan, trastuzumab, tretinoin, vinblastine, vincristine, vindesine, vinorelbine, or a combination thereof. Preferably, the chemotherapeutic comprises a platinum-based chemotherapeutic, such as cisplatin, oxaliplatin, satraplatin, and carboplatin, most preferably cisplatin.

Many combinations of chemotherapeutic drugs have been developed for the treatment of cancer. Examples of such combinations of chemotherapeutic drugs to which HDAC inhibitors may be added are included in Table 1.

TABLE 1
Exemplary combinations of chemotheraputic drugs.

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, Procarbazine, |
| | Prednisone |
| BEACOPP | Bleomycin, Etoposide, Doxorubicin, Cyclophosphamide, |
| | Vincristine, Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, Prednisone, |
| | Asparaginase |
| CAMP | Cyclophosphamide, Doxorubicin, Methotrexate, |
| | Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or without/ |
| | Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, |
| | Doxorubicin, Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, Prednisone |
| CMFVP | Cyclophosphamide, Methotrexate, Fluorouracil, Vincristine, |
| | Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, Leucovorin, |
| | Cytarabine |
| COMP | Cyclophosphamide, Vincristine, Methotrexate, Prednisone |
| Cooper Regimen | Cyclophosphamide, Methotrexate, Fluorouracil, Vincristine, |
| | Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP(Chronic lymphocytic | Chlorambucil, Prednisone |
| leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, Fluorouracil |
| ICE-T | Ifosfamide, Carboplatin, Etoposide, Paclitaxel, Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, |
| | Melphalan |
| MAC-III | Methotrexate, Leucovorin, Dactinomycin, |
| | Cyclophosphamide |
| MACC | Methotrexate, Doxorubicin, Cyclophosphamide, Lomustine |
| MACOP-B | Methotrexate, Leucovorin, Doxorubicin, Cyclophosphamide, |
| | Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, |
| | Doxorubicin, Bleomycin, Vinblastine |
| MP (multiple myeloma) | Melphalan, Prednisone |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, |
| | Cytarabine, Bleomycin, Vincristine, Methotrexate, |
| | Leucovorin, Cotrimoxazole |
| PRoMACE/MOPP | Prednisone, Doxorubicin, Cyclophosphamide, Etoposide, |
| | Mechlorethamine, Vincristine, Procarbazine, Methotrexate, |
| | Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, |
| | Bleomycin, Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, |
| | Bleomycin |
| VAC | Vincristine, Dactinomycin, Cyclophosphamide |
| VACAdr | Vincristine, Cyclophosphamide, Doxorubicin, Dactinomycin, |
| | Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, |
| | Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or |
| | Mitoxantrone |
| “8 in 1” | Methylprednisolone, Vincristine, Lomustine, Procarbazine, |
| | Hydroxyurea, Cisplatin, Cytarabine, Dacarbazine |

In certain embodiments, a combination therapy disclosed herein may be combined with non-chemical methods of cancer treatment, such as radiation therapy, surgery, thermoablation, focused ultrasound therapy, cryotherapy, or any combination of these.

It will be understood by one of ordinary skill in the art that the compositions and methods described herein may be adapted and modified as is appropriate for the application being addressed and that the compositions and methods described herein may be employed in other suitable applications, and that such other additions and modifications will not depart from the scope hereof. The disclosure contemplates all uses of the compounds and compositions of the disclosure, including their use in therapeutic methods, in diagnostic assays, and their use as research tools.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

The term “chemotherapeutic” refers to one or more drugs, especially one or more anti-cancer drugs.

The term “combination treatment” refers to a treatment wherein a HDAC inhibitor, e.g., AR-42, is administered conjointly with another chemotherapeutic, e.g., cisplatin. An effective amount of each agent may be an amount of the agent that is less than the effective amount of that agent when administered without the other. The HDAC inhibitor and the other chemotherapeutic may be administered simultaneously or sequentially.

The term “compound” refers to chemical compounds, which may be either HDAC inhibitors or chemotherapeutic drugs.

The terms “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g. solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapies such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulations, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

A “therapeutically effective amount” (“effective amount”) or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer. The skilled worker can readily determine the effective amount for a given situation by routine experimentation.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. The selection of the appropriate salt will be known to one skilled in the art.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

In some embodiments, the invention relates to a method wherein administering the chemotherapeutic comprises administering a low-dose chemotherapy. In some embodiments, the chemotherapeutic is administered at an amount that is less than the standard amount. Administering a chemotherapeutic at a standard amount refers to administering the chemotherapeutic according to the chemotherapeutic's label or pursuant to professional guidelines, such as the National Comprehensive Cancer Network guidelines. For example, a standard amount of cisplatin for treating advanced bladder cancer is 50 to 70 mg/m² per cycle once every 3 to 4 weeks. In some embodiments, the chemotherapeutic is cisplatin, the cancer is advanced bladder cancer, and cisplatin is administered at an amount that is less than 50 mg/m² per cycle, such as 40 mg/m² per cycle, 30 mg/m² per cycle, 20 mg/m² per cycle, or 10 mg/m² per cycle or less. A standard amount of gemcitabine for treating bladder cancer in combination with cisplatin includes administering 1000 mg/m² of gemcitabine on 2 days of a two-week cycle or on 3 days of a three-week cycle. In some embodiments, the chemotherapeutic is gemcitabine, the cancer is bladder cancer, and gemcitabine is administered at an amount that is less than 1000 mg/m², for example, at 900 mg/m², 800 mg/m², 700 mg/m², 600 mg/m², or 500 mg/m² or less. In some embodiments, the chemotherapeutic is administered at an amount that reduces the risk or severity of side effects associated with the chemotherapeutic relative to the standard amount. In some embodiments, the patient has a disease or condition that is a contraindication for standard-dose chemotherapy. In some embodiments, the patient has reduced creatinine clearance or neutropenia. In some embodiments, the combination of administering the histone-deacetylase inhibitor and the chemotherapeutic is more efficacious than administering either treatment alone (additive), or even synergistic.

In some embodiments, the invention relates to a method wherein the histone-deacetylase inhibitor increases apoptosis or autophagy of cancer cells. In some embodiments, the subject is a mammal. In some embodiments, the subject is a human.

This disclosure will be better understood from the Experimental Details which follow. However, one skilled in the art will readily appreciate that the specific methods and results discussed are merely illustrative of the disclosure as described more fully in the embodiments which follow thereafter.

### EXEMPLIFICATION

### Example 1

### Methods

Bladder Cancer Cell Lines—SW780 and HT1376 cell lines were grown in RPMI with 10% fetal bovine serum at 37° C. with 5% CO₂ and treated with cisplatin, AR-42, sodium butyrate, trichostatin A, and valproic acid at concentrations indicated.

Tumor Model—SW780 bladder cancer cells were combined with fetal bladder mesenchymal cells in the presence of Matrigel and implanted subcutaneously in NOD scid IL-2 receptor gamma chain knockout animals.

Flow Cytometry—Cells were analyzed on FACS LSRII and sorted on FACSAria (BD Biosciences) and analyzed using CellQuest. Antibodies including anti-CD44 and anti-CD49f antibodies were obtained from BD Biosciences.

Apoptosis and Autophagy Analysis—Apoptosis was analyzed by staining with propidium iodide and annexin V, while autophagy was assessed by staining with LC3 and analyzed by flow cytometry.

### Example 2

### Bladder Cancer Cell Line Response to Cisplatin and HDAC Inhibitors

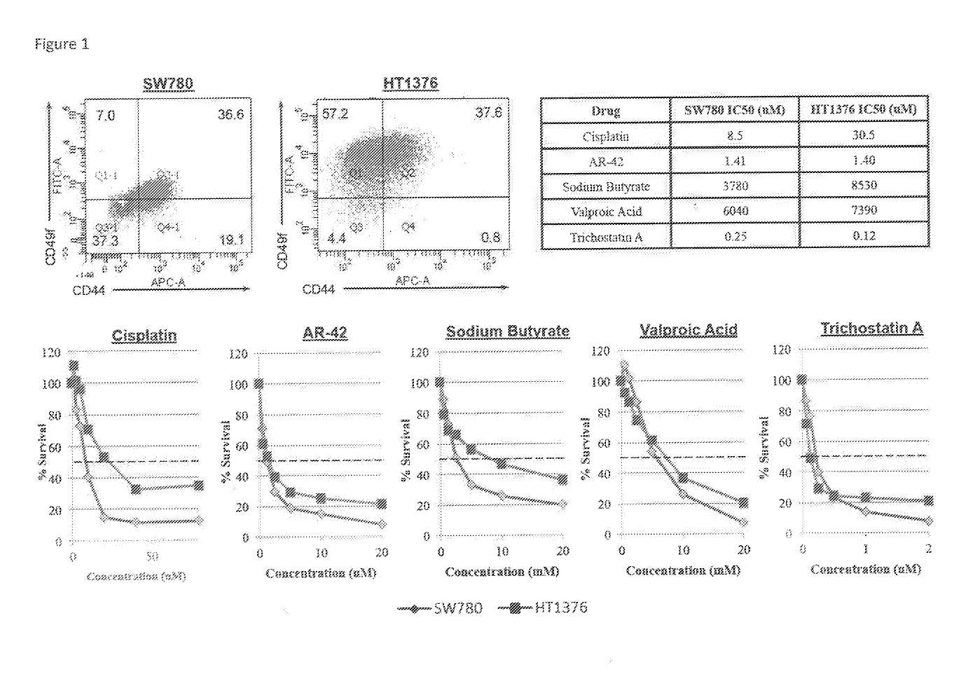

SW780 and HT1376 cell lines were treated with cisplatin, AR-42, sodium butyrate, valproic acid, and trichostatin A at various concentrations and cell viability was determined by MTT incorporation (FIG. 1). The IC50s for the compounds are reported in Table 2 below.

TABLE 2

| Drug | SW780 IC50 (uM) | HT1376 IC50 (uM) |
|---|---|---|
| Cisplatin | 8.5 | 30.5 |
| AR-42 | 1.41 | 1.40 |
| Sodium Butyrate | 3780 | 8530 |
| Valproic Acid | 6040 | 7390 |
| Trichostatin A | 0.25 | 0.12 |

### Example 3

### Cisplatin Synergy with AR-42

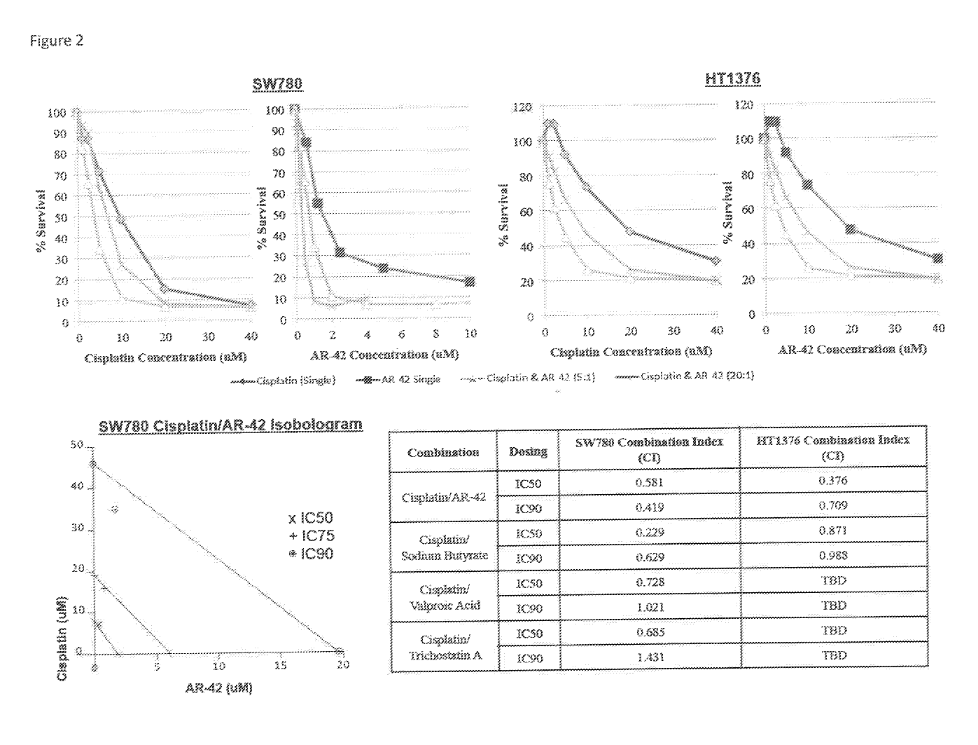

SW780 and HT1376 cells treated with cisplatin, AR-42, a combination cisplatin and AR-42 at a 5:1 ratio, a combination cisplatin and AR-42 in a 20:1 molar ratio, as assessed by MTT incorporation (FIG. 2). Each compound displayed synergy with cisplatin when administered at its IC50 (Table 3). AR-42 and Sodium Butyrate displayed synergy with cisplatin when administered at their IC90s.

TABLE 3

| | | SW780 Combina- | HT1376 Combina- |
|---|---|---|---|
| Combination | Dosing | tion Index (CI) | tion Index (CI) |
| Cisplatin/AR- | IC50 | 0.581 | 0.376 |
| 42 | IC90 | 0.419 | 0.709 |
| Cisplatin/ | IC50 | 0.229 | 0.871 |
| Sodium Butyrate | IC90 | 0.629 | 0.988 |
| Cisplatin/ | IC50 | 0.728 | TBD |
| Valproic Acid | IC90 | 1.021 | TBD |
| Cisplatin/ | IC50 | 0.665 | TBD |
| Trichostatin A | IC90 | 1.431 | TBD |

### Example 4

### Synergy of AR-42 and Cisplatin in CD44⁺CD49f⁺ Bladder Cancer Cells

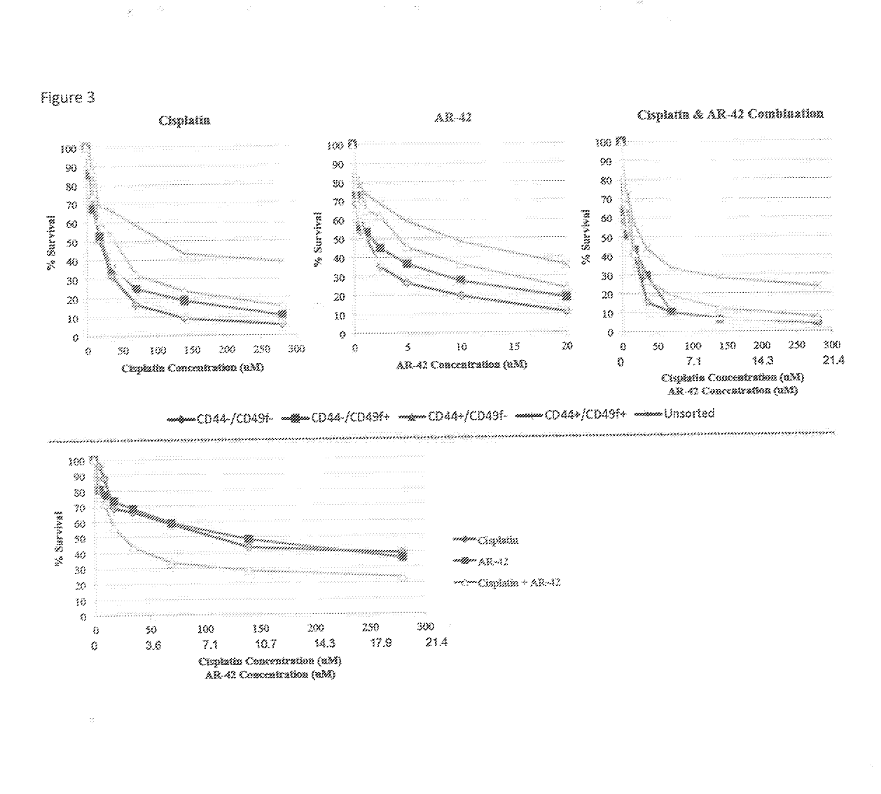

SW780 bladder cancer cells that were sorted into CD44⁻CD49f⁻, CD44⁺CD49f⁻, CD44⁻CD49f⁺, and CD44⁺CD49f⁺ populations by flow cytometry and treated with cisplatin, AR-42, or a combination cisplatin and AR-42. Cell survival was assessed by MTT incorporation. The combination of cisplatin and AR-42 was more effective at killing CD44⁻CD49f⁺ cancer stem cells than either treatment alone (FIG. 3).

### Example 5

### Sequential Administration of AR-42 and Cisplatin in vitro

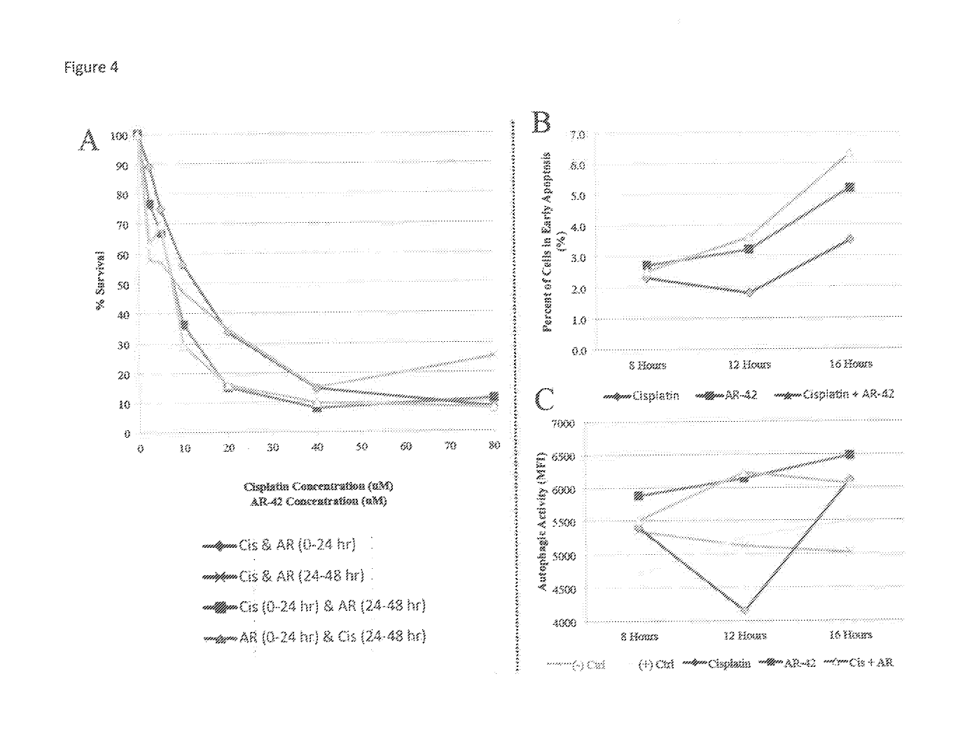

SW780 cells were treated with cisplatin and AR-42 simultaneously for 24 hours, sequentially with cisplatin for 24 hours followed by AR-42 for 24 hours, and sequentially with AR-42 for 24 hours followed by cisplatin for 24 hours (FIG. 4). Flow cytometry was used to assess apoptosis, by Annexin V staining, and autophagy, by LC3 staining.

### Example 6

### In vivo Administration of Combined Cisplatin and AR-42 Decreases Tumor Size Relative to Cisplatin or AR-42 Alone

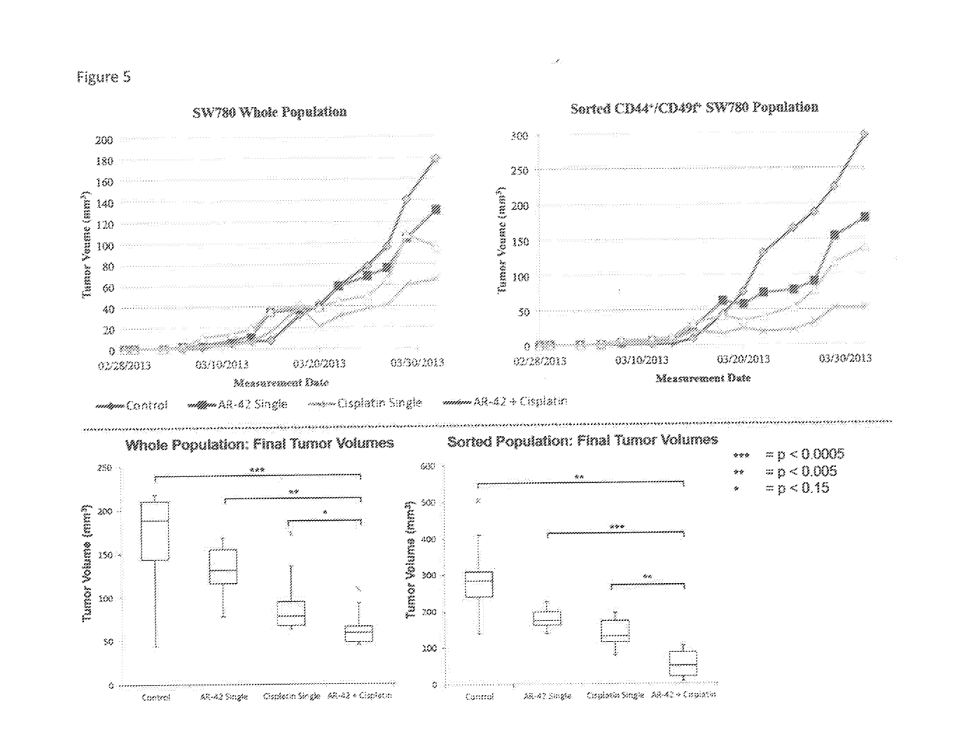

A composition comprising 10⁵ SW780 cells and 10⁶ human fetal bladder mesenchymal cells in Matrigel were subcutaneously implanted in Nod scid IL-2 receptor gamma knockout mice. The mice were administered either AR-42, cisplatin, or a combination treatment comprising both AR-42 and cisplatin. The tumor sizes for mice that received the combination treatment were significantly smaller than mice that received AR-42 or cisplatin alone (FIG. 5).

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating cancer in a subject in need thereof, comprising administering to the subject an effective amount of a histone-deacetylase inhibitor and a chemotherapeutic.

2. The method of claim 1, wherein the histone-deacetylase inhibitor is administered orally.

3. The method of claim 1, wherein the histone-deacetylase inhibitor is AR-42.

4. The method of claim 1, wherein administering a chemotherapeutic comprises administering aminoglutethimide, amsacrine, anastrozole, asparaginase, bcg, bicalutamide, bleomycin, bortezomib, buserelin, busulfan, campothecin, capecitabine, carboplatin, carfilzomib, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, lenalidomide, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pemetrexed, pentostatin, perifosine, plicamycin, pomalidomide, porfimer, procarbazine, raltitrexed, rituximab, sorafenib, streptozocin, sunitinib, suramin, tamoxifen, temozolomide, temsirolimus, teniposide, testosterone, thalidomide, thioguanine, thiotepa, titanocene dichloride, topotecan, trastuzumab, tretinoin, vinblastine, vincristine, vindesine, or vinorelbine.

5. The method of claim 4, wherein administering a chemotherapeutic comprises administering cisplatin or pemetrexed.

6. The method of claim 5, wherein administering a chemotherapeutic comprises administering cisplatin.

7. The method of claim 1, wherein the cancer is ovarian cancer, cervical cancer, brain cancer, lung cancer, skin cancer, colorectal cancer, esophageal cancer, breast cancer, prostate cancer, leukemia, lymphoma, multiple myeloma, bone cancer, pancreatic cancer, bladder cancer, endometrial cancer, kidney cancer, liver cancer, eye cancer, pituitary cancer, testicular cancer, and stomach cancer.

8. The method of claim 7, wherein the cancer is bladder cancer, lung cancer, breast cancer, prostate cancer, lymphoma, or leukemia.

9. The method of claim 8, wherein the cancer is bladder cancer.

10. The method of claim 1, wherein administering a chemotherapeutic comprises administering a low-dose chemotherapy.

11. The method of claim 1, wherein the chemotherapeutic is administered at an amount that is less than the standard amount.

12. The method of claim 10, wherein the chemotherapeutic is administered at an amount that reduces the risk or severity of side effects associated with the chemotherapeutic relative to the standard amount.

13. The method of claim 10, wherein the patient has a disease or condition that is a contraindication for standard-dose chemotherapy.

14. The method of claim 13, wherein the patient has reduced creatinine clearance or neutropenia.

15. The method of claim 1, wherein the combination of administering the histone-deacetylase inhibitor and the chemotherapeutic is more efficacious than administering either treatment alone.

16. The method of claim 1, wherein the histone-deacetylase inhibitor increases the apoptosis or autophagy of cancer cells.

17. The method of claim 1, wherein the subject is a mammal.

18. The method of claim 17, wherein the subject is a human.

19. A pharmaceutical composition comprising a histone-deacetylase inhibitor and a chemotherapeutic.

20. The pharmaceutical composition of claim 19, wherein the pharmaceutical composition is for oral administration.
